(19) United States
(12) Patent Application Publication
(10) Pub. No.: US 2013/0117891 A1
(43) Pub. Date: May 9, 2013

(54) GROWTH PROMOTING FUSION PROTEINS

(71) Applicants: Dirk G Inzé, Aalst (BE); Nathalie Gonzalez, Merelbeke (BE); William M. Gray, St. Paul, MN (US); Angela K. Spartz, Falcon Heights, MN (US)

(72) Inventors: Dirk G Inzé, Aalst (BE); Nathalie Gonzalez, Merelbeke (BE); William M. Gray, St. Paul, MN (US); Angela K. Spartz, Falcon Heights, MN (US)

(21) Appl. No.: 13/697,321

(22) PCT Filed: May 11, 2011
(86) PCT No.: PCT/EP2011/057589

§ 371 Date: Jan. 18, 2013

Related U.S. Application Data
(60) Provisional application No. 61/395,398, filed on May 11, 2010.

(30) Foreign Application Priority Data
May 11, 2010 (GB) 1007834.3

(51) Int. Cl. C07K 14/415

(52) U.S. Cl. CPC C07K 14/415
USPC 800/290; 530/370; 800/298; 800/306; 800/312; 800/314; 800/320; 800/320.3; 800/320.2; 800/320.1

(57) ABSTRACT

The present invention relates to fusion proteins that promote plant growth. More specifically, it relates to fusion proteins of polypeptides of the SAUR family fused to a heterologous polypeptide, preferably fused at the N-terminal end of the SAUR polypeptide. Said polypeptide can be expressed in a transgenic plant, possible in combination with other recombinant genes, to obtain an additive or even synergistic effect.

The present invention relates to fusion proteins that promote plant growth. More specifically, it relates to fusion proteins of polypeptides of the SAUR family fused to a heterologous polypeptide, preferably fused at the N-terminal end of the SAUR polypeptide. Said polypeptide can be expressed in a transgenic plant, possible in combination with other recombinant genes, to obtain an additive or even synergistic effect.

The demand for more plant derived products has spectacularly increased. In the near future the challenge for agriculture will be to fulfill the growing demands for feed and food in a sustainable manner. Moreover plants start to play an important role as energy sources. To cope with these major challenges, a profound increase in plant yield will have to be achieved. Biomass production is a multi-factorial system in which a plethora of processes are fed into the activity of meristems that give rise to new cells, tissues, and organs. Although a considerable amount of research on yield performance is being performed little is known about the molecular networks underpinning yield (Van Camp, 2005; Gonzalez et a1.2009). Many genes have been described in Arabidopsis thaliana that, when mutated or ectopically expressed, result in the formation of larger structures, such as leaves or roots. However, notwithstanding extensive research, the effect of overexpression of a gene, and especially the effect of expression of combinations of genes is unpredictable. There is still need for further genes that can be used to increase yield, especially genes that have an additive or even synergetic effect with other growth promoting genes.

The small auxin-up RNA (SAUR) family comprises a large set of genes whose expressions are early auxin-responsive (Franco et al., 1990; Anai et al., 1998; Jain et al., 2006). However, the function of these genes is largely unknown. Knauss et al. (2002) indicate that SAUR2 of Zea mays encodes a short lived nuclear protein that might be involved in auxin-mediated cell elongation. Recently, Kant et al. (2009) demonstrated that SAUR 39 of Oryza sativa acts as a negative regulator of auxin synthesis and transport in rice. WO2008061240 discloses the use of SAUR22 of Arabidopsis thaliana and other members of the SAUR family to improve cold tolerance; however, no data are shown.

Surprisingly we found that expression of a SAUR fusion protein, but not overexpression of SAUR itself is promoting plant growth. Even more surprisingly only N-terminal fusions to SAUR, and not the C-terminal fusion show the plant growth promoting effect. Interestingly, the growth promoting effect of SAUR can be combined with the effect of other growth promoting genes, resulting in an additive or even synergetic effect on plant growth. This effect is rather exceptional, as most combinations of growth promoting genes give a less than additive effect, or even a smaller effect for the combination of genes than for each individual growth promoting gene.

A first aspect of the invention is a fusion protein comprising a SAUR polypeptide and a heterologous polypeptide. SAUR proteins are known to the person skilled in the art, and include but are not limited to the genes listed in table 1, and homologues or orthologues thereof. The terms “polypeptide” and “protein” are used interchangeably herein and refer to amino acids in a polymeric form of any length, linked together by peptide bonds. A “fusion protein” refers to a protein wherein two polypeptides which in nature do not occur together as part of the same protein are linked to each other by a peptide bond to form one protein. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. Preferably, said homologue, orthologue or paralogue has a sequence identity at protein level of at least 40%, 41%, 42%, 43%, 44%, 45%, 46%, 47%, 48%, 49%, preferably 50%, 51%, 52%, 53%, 54% or 55%, 56%, 57%, 58%, 59%, preferably 60%, 61%, 62%, 63%, 64%, 65%, 66%, 67%, 68%, 69%, more preferably 70%, 71%, 72%, 73%, 74%, 75%, 76%, 77%, 78%, 79%, even more preferably 80%, 81%, 82%, 83%, 84%, 85%, 86%, 87%, 88%, 89% most preferably 90% 91%, 92%, 93%, 94%, 95%, 96%, 97%, 98%, 99% or more as measured in a BLASTp (Altschul et al., 1997; Altschul et al., 2005). Preferably, said SAUR polypeptide comprises the sequences (E/K/R) G (H/N/FN/S) (V/L/F) (P/A) V (Y/S/C) V (SEQ ID NO 5) and/or L L (R/E/K/S) (R/K/E/D) (NV) (NE) (Q/E) E (Y/F) G (Y/F) (SEQ ID NO 6), more preferably said SAUR polypeptide comprises both sequences. Even more preferably, said SAUR polypeptide is selected from the group consisting of SAUR 19, SAUR 21, SAUR 23 and SAUR24. Most preferably, said SAUR polypeptide comprises a sequence selected from the group consisting of SEQ ID NO 1-4 (sequences tested). Preferably, the fusion protein according to the invention is a fusion protein wherein said heterologous polypeptide is fused to the N-terminal end of said SAUR polypeptide.

Another aspect of the invention is the use of a fusion protein according to the invention to increase plan growth and/or plant yield. Increase of plant growth and/or yield is measured by comparing the test plant, comprising a gene used according to the invention, with the parental, non-transformed plant, grown under the same conditions as control. Preferably, increase of growth is measured as an increase of biomass production. “Yield” refers to a situation where only a part of the plant, preferably an economical important part of the plant, such as the leaves, roots or seeds, is increased in biomass. The term “increase” as used here means least a 5%, 6%, 7%, 8%, 9% or 10%, preferably at least 15% or 20%, more preferably 25%, 30%, 35% or 40% more yield and/or growth in comparison to control plants as defined herein. Increase of plant growth, as used here, is preferably measured as increase of any one or more of total plant biomass, leaf biomass, root biomass and seed biomass. In one preferred embodiment, said increase is an increase in total plant biomass. Preferably, said use is the expression of the fusion protein in said plant. Preferably, said plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis. In a preferred embodiment, said plant is a crop plant, preferably a monocot or a cereal, even more preferably it is a cereal selected from the group consisting of rice, maize, wheat, barley, millet, rye, sorghum and oats.

Still another aspect of the invention is the use of a fusion protein according to the invention, wherein said fusion protein is expressed in combination with a protein selected from the group consisting of ARL (genbank accession number: NP_{—}850409.1), ANT(AAB17364.1), AGF1(NP_{—}195265.2), APC10(Q9ZPW2.2), GRF5(NP_{—}568325.1) and AVP1(NP_{—}001077542.1), in combination with the expression of the JAW gene encoding a microRNA (AY922344.1), and/or in combination with a downregulated or inactivated gene encoding DA1-1 (NP_{—}173361.1), preferably a DA1-1 knock out, or in the respective combination with or a homologue or orthologue of said genes. An inactivated DA1-1 gene as used here refers also to DA1-1 genes encoding a defective, or less active DA1-1 protein. Preferably the increase or plant growth and/or yield, obtained by the coexpression of the genes, is at least additive, preferably said increase is synergetic.

Still another aspect of the invention is a transgenic plant, comprising a fusion protein according to the invention. For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either (a) the nucleic acid sequences encoding proteins useful in the methods of the invention, or (b) genetic control sequence(s) which is operably linked with the nucleic acid sequence according to the invention, for example a promoter, or (c) a) and b) are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815. The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. In one preferred embodiment, the transgenic plant according to invention further comprising a recombinant gene encoding a protein selected from the group consisting of ARL, ANT, AGF1, APC10, GRF5 and AVP1, or a homologue or orthologue thereof. In another preferred embodiment, the transgenic plant is comprising a downregulated DA1-1 gene, preferably a DA1-1 knock out, or a downregulation or knock out of a homologue or orthologue of DA1-1. In still another preferred embodiment, the transgenic plant is further comprising a recombinant JAW gene, encoding a microRNA. Preferably said transgenic plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis. In a preferred embodiment, said plant is a crop plant, preferably a monocot or a cereal, even more preferably it is a cereal selected from the group consisting of rice, maize, wheat, barley, millet, rye, sorghum and oats. Another aspect of the invention is a method for obtaining plants with increased growth characteristics, comprising (1) isolating a nucleic acid encoding a polypeptide of the SAUR family, (2) fusing said nucleic acid at the 5′ end to a nucleic acid encoding a heterologous peptide, wherein said fusion results in a N-terminal fusion to the SAUR polypeptide (3) transforming the fused nucleic acid into a plant. Preferably, said fused nucleic acid is operably linked to a suitable promoter. “Operably linked” refers to a juxtaposition wherein the components so described are in a relationship permitting them to function in their intended manner. A promoter sequence “operably linked” to a coding sequence is ligated in such a way that expression of the coding sequence is achieved under conditions compatible with the promoter sequence. Preferably, said plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis. In a preferred embodiment, said plant is a crop plant, preferably a monocot or a cereal, even more preferably it is a cereal selected from the group consisting of rice, maize, wheat, barley, millet, rye, sorghum and oats. Preferably, said SAUR polypeptide comprises the sequences (EKR) G (HNFYS) (VLF) (PA) V (YSC) V and/or L L (REKS) (RKED) (AV) (AE) (QE) E (YF) G (YF), more preferably said SAUR polypeptide comprises both sequences. More preferably, said SAUR polypeptide comprises the conserved domain pfam02519 (http://www.ncbi.nlm.nih.gov/Structure/cdd/cddsrv.cgi?uid=145583; Marchler-Bauer et al., 2009). Even more preferably, said SAUR polypeptide is selected from the group consisting of SAUR 19, SAUR 21, SAUR 23 and SAUR24. Most preferably, said SAUR polypeptide comprises a sequence selected from the group consisting of SEQ ID NO 1-4 (sequences tested). Preferably, the fusion protein according to the invention is a fusion protein wherein said heterologous polypeptide is fused to the N-terminal end of said SAUR polypeptide.

TABLE I
non-limiting list of members of the SAUR family

| Species | length | accession number | |
|---|---|---|---|
| SAUR [Raphanus sativus] | 95 aa | BAA25434.1 | GI: 3043536 |
| SAUR family protein [Musa balbisiana] | 176 aa | BAG70988.1 | GI: 199601729 |
| SAUR family protein [Musa balbisiana] | 176 aa | BAG70998.1 | GI: 199601704 |
| SAUR33 [Zea mays] | 131 aa | NP_001159042.1 | GI: 259490681 |
| SAUR12 [Zea mays] | 125 aa | NP_001151012.1 | GI: 226533132 |
| SAUR55 [Zea mays] | 149 aa | NP_001148334.1 | GI: 226531724 |
| SAUR52 [Zea mays] | 152 aa | NP_001151993.1 | GI: 226530832 |
| SAUR20 [Zea mays] | 166 aa | NP_001148938.1 | GI: 226530568 |
| SAUR37 [Zea mays] | 157 aa | NP_001147560.1 | GI: 226529857 |
| SAUR40 [Zea mays] | 89 aa | NP_001152402.1 | GI: 226528649 |
| SAUR31 [Zea mays] | 102 aa | NP_001148413.1 | GI: 226510187 |
| SAUR23 [Zea mays] | 161 aa | NP_001151649.1 | GI: 226510085 |
| SAUR25 [Zea mays] | 139 aa | NP_001148237.1 | GI: 226509128 |
| SAUR20 [Zea mays] | 169 aa | NP_001150569.1 | GI: 226507274 |
| SAUR30 [Zea mays] | 117 aa | NP_001151597.1 | GI: 226506882 |
| SAUR11 [Zea mays] | 202 aa | NP_001151006.1 | GI: 226506308 |
| SAUR16 [Zea mays] | 108 aa | NP_001147394.1 | GI: 226505984 |
| SAUR11 [Zea mays] | 199 aa | NP_001151756.1 | GI: 226505124 |
| SAUR1 [Zea mays] | 103 aa | NP_001152203.1 | GI: 226504722 |
| SAUR52 [Zea mays] | 147 aa | NP_001150183.1 | GI: 226503087 |
| SAUR14 [Zea mays] | 103 aa | NP_001147398.1 | GI: 226501774 |
| SAUR56 [Zea mays] | 127 aa | NP_001150182.1 | GI: 226499078 |
| SAUR33 [Zea mays] | 137 aa | NP_001146880.1 | GI: 226494413 |
| SAUR9 [Zea mays] | 107 aa | NP_001147174.1 | GI: 226492799 |
| SAUR family protein [Populus trichocarpa] | 118 aa | EEF06097.1 | GI: 222868966 |
| SAUR family protein [Populus trichocarpa] | 125 aa | EEF05238.1 | GI: 222868107 |
| SAUR family protein [Populus trichocarpa] | 123 aa | EEF05232.1 | GI: 222868101 |
| SAUR family protein [Populus trichocarpa] | 144 aa | EEF04069.1 | GI: 222866938 |
| SAUR family protein [Populus trichocarpa] | 160 aa | EEF03893.1 | GI: 222866762 |
| SAUR family protein [Populus trichocarpa] | 119 aa | EEF03856.1 | GI: 222866725 |
| SAUR family protein [Populus trichocarpa] | 128 aa | EEF03855.1 | GI: 222866724 |
| SAUR family protein [Populus trichocarpa] | 148 aa | EEF03854.1 | GI: 222866723 |
| SAUR family protein [Populus trichocarpa] | 167 aa | EEF03289.1 | GI: 222866158 |
| SAUR family protein [Populus trichocarpa] | 153 aa | EEF03223.1 | GI: 222866092 |
| SAUR family protein [Populus trichocarpa] | 185 aa | EEF02601.1 | GI: 222865470 |
| SAUR family protein [Populus trichocarpa] | 131 aa | EEF02550.1 | GI: 222865419 |
| SAUR family protein [Populus trichocarpa] | 156 aa | EEF01541.1 | GI: 222864410 |
| SAUR family protein [Populus trichocarpa] | 121 aa | EEF01490.1 | GI: 222864359 |
| SAUR family protein [Populus trichocarpa] | 177 aa | EEF00366.1 | GI: 222862859 |
| SAUR family protein [Populus trichocarpa] | 110 aa | EEE98534.1 | GI: 222860992 |
| SAUR family protein [Populus trichocarpa] | 170 aa | EEE98363.1 | GI: 222860821 |
| SAUR family protein [Populus trichocarpa] | 125 aa | EEE98233.1 | GI: 222860686 |
| SAUR family protein [Populus trichocarpa] | 181 aa | EEE96973.1 | GI: 222859426 |
| SAUR family protein [Populus trichocarpa] | 135 aa | EEE96685.1 | GI: 222859138 |
| SAUR family protein [Populus trichocarpa] | 97 aa | EEE95603.1 | GI: 222858056 |
| SAUR family protein [Populus trichocarpa] | 104 aa | EEE94704.1 | GI: 222857157 |
| SAUR family protein [Populus trichocarpa] | 149 aa | EEE93913.1 | GI: 222856366 |
| SAUR family protein [Populus trichocarpa] | 123 aa | EEE93715.1 | GI: 222856168 |
| SAUR family protein [Populus trichocarpa] | 136 aa | EEE93401.1 | GI: 222855854 |
| SAUR family protein [Populus trichocarpa] | 124 aa | EEE92944.1 | GI: 222855397 |
| SAUR family protein [Populus trichocarpa] | 149 aa | EEE91612.1 | GI: 222854065 |
| SAUR family protein [Populus trichocarpa] | 104 aa | EEE91306.1 | GI: 222853759 |
| SAUR family protein [Populus trichocarpa] | 114 aa | EEE90949.1 | GI: 222853402 |
| SAUR family protein [Populus trichocarpa] | 138 aa | EEE90544.1 | GI: 222852997 |
| SAUR family protein [Populus trichocarpa] | 136 aa | EEE89259.1 | GI: 222851712 |
| SAUR family protein [Populus trichocarpa] | 132 aa | EEE88450.1 | GI: 222850903 |
| SAUR family protein [Populus trichocarpa] | 146 aa | EEE87704.1 | GI: 222850157 |
| SAUR family protein [Populus trichocarpa] | 98 aa | EEE87702.1 | GI: 222850155 |
| SAUR family protein [Populus trichocarpa] | 68 aa | EEE87701.1 | GI: 222850154 |
| SAUR family protein [Populus trichocarpa] | 141 aa | EEE87700.1 | GI: 222850153 |
| SAUR family protein [Populus trichocarpa] | 104 aa | EEE86957.1 | GI: 222849410 |
| SAUR family protein [Populus trichocarpa] | 91 aa | EEE86955.1 | GI: 222849408 |
| SAUR family protein [Populus trichocarpa] | 92 aa | EEE86954.1 | GI: 222849407 |
| SAUR family protein [Populus trichocarpa] | 99 aa | EEE86953.1 | GI: 222849406 |
| SAUR family protein [Populus trichocarpa] | 98 aa | EEE86951.1 | GI: 222849404 |
| SAUR family protein [Populus trichocarpa] | 145 aa | EEE86950.1 | GI: 222849403 |
| SAUR family protein [Populus trichocarpa] | 93 aa | EEE86949.1 | GI: 222849402 |
| SAUR family protein [Populus trichocarpa] | 95 aa | EEE86948.1 | GI: 222849401 |
| SAUR family protein [Populus trichocarpa] | 97 aa | EEE86947.1 | GI: 222849400 |
| SAUR family protein [Populus trichocarpa] | 65 aa | EEE86946.1 | GI: 222849399 |
| SAUR family protein [Populus trichocarpa] | 90 aa | EEE86945.1 | GI: 222849398 |
| SAUR family protein [Populus trichocarpa] | 107 aa | EEE86944.1 | GI: 222849397 |
| SAUR family protein [Populus trichocarpa] | 149 aa | EEE86883.1 | GI: 222849336 |
| SAUR family protein [Populus trichocarpa] | 148 aa | EEE86882.1 | GI: 222849335 |
| SAUR family protein [Populus trichocarpa] | 148 aa | EEE86881.1 | GI: 222849334 |
| SAUR family protein [Populus trichocarpa] | 148 aa | EEE86880.1 | GI: 222849333 |
| SAUR family protein [Populus trichocarpa] | 146 aa | EEE86879.1 | GI: 222849332 |
| SAUR family protein [Populus trichocarpa] | 148 aa | EEE86729.1 | GI: 222849182 |
| SAUR family protein [Populus trichocarpa] | 74 aa | EEE86671.1 | GI: 222849124 |
| SAUR family protein [Populus trichocarpa] | 89 aa | EEE86670.1 | GI: 222849123 |
| SAUR family protein [Populus trichocarpa] | 72 aa | EEE86669.1 | GI: 222849122 |
| SAUR family protein [Populus trichocarpa] | 92 aa | EEE86668.1 | GI: 222849121 |
| SAUR family protein [Populus trichocarpa] | 97 aa | EEE86667.1 | GI: 222849120 |
| SAUR family protein [Populus trichocarpa] | 67 aa | EEE86666.1 | GI: 222849119 |
| SAUR family protein [Populus trichocarpa] | 90 aa | EEE86665.1 | GI: 222849118 |
| SAUR family protein [Populus trichocarpa] | 94 aa | EEE86664.1 | GI: 222849117 |
| SAUR family protein [Populus trichocarpa] | 99 aa | EEE86663.1 | GI: 222849116 |
| SAUR family protein [Populus trichocarpa] | 99 aa | EEE86662.1 | GI: 222849115 |
| SAUR family protein [Populus trichocarpa] | 96 aa | EEE86661.1 | GI: 222849114 |
| SAUR family protein [Populus trichocarpa] | 98 aa | EEE86660.1 | GI: 222849113 |
| SAUR family protein [Populus trichocarpa] | 104 aa | EEE86658.1 | GI: 222849111 |
| SAUR family protein [Populus trichocarpa] | 91 aa | EEE85987.1 | GI: 222848440 |
| SAUR family protein [Populus trichocarpa] | 98 aa | EEE85986.1 | GI: 222848439 |
| SAUR family protein [Populus trichocarpa] | 101 aa | EEE85985.1 | GI: 222848438 |
| SAUR family protein [Populus trichocarpa] | 145 aa | EEE85982.1 | GI: 222848435 |
| SAUR family protein [Populus trichocarpa] | 106 aa | EEE85019.1 | GI: 222847472 |
| SAUR family protein [Populus trichocarpa] | 160 aa | EEE84134.1 | GI: 222846587 |
| SAUR family protein [Populus trichocarpa] | 127 aa | EEE83807.1 | GI: 222846260 |
| SAUR family protein [Populus trichocarpa] | 150 aa | EEE81433.1 | GI: 222843886 |
| SAUR family protein [Populus trichocarpa] | 149 aa | EEE81260.1 | GI: 222843713 |
| SAUR family protein [Populus trichocarpa] | 100 aa | EEE81154.1 | GI: 222843607 |
| SAUR family protein [Populus trichocarpa] | 111 aa | EEE80702.1 | GI: 222843155 |
| SAUR family protein [Populus trichocarpa] | 171 aa | EEE80545.1 | GI: 222842998 |
| SAUR family protein [Populus trichocarpa] | 94 aa | EEE80141.1 | GI: 222842594 |
| SAUR family protein [Populus trichocarpa] | 104 aa | EEE79990.1 | GI: 222842443 |
| SAUR family protein [Populus trichocarpa] | 126 aa | EEE79223.1 | GI: 222841676 |
| SAUR family protein [Populus trichocarpa] | 167 aa | EEE78749.1 | GI: 222841202 |
| SAUR family protein [Populus trichocarpa] | 169 aa | EEE78501.1 | GI: 222840954 |
| SAUR family protein [Populus trichocarpa] | 106 aa | EEE78304.1 | GI: 222840757 |
| SAUR protein [Medicago truncatula] | 125 aa | ABE80127.1 | GI: 92870927 |
| SAUR protein [Medicago truncatula] | 140 aa | ABD32679.1 | GI: 87240821 |
| SAUR protein [Medicago truncatula] | 130 aa | ABD32678.1 | GI: 87240820 |
| SAUR family protein [Populus trichocarpa] | 109 aa | XP_002337465.1 | GI: 224154353 |
| SAUR family protein [Populus trichocarpa] | 144 aa | XP_002336416.1 | GI: 224147130 |
| SAUR family protein [Populus trichocarpa] | 177 aa | XP_002325984.1 | GI: 224146373 |
| SAUR family protein [Populus trichocarpa] | 167 aa | XP_002324724.1 | GI: 224142769 |
| SAUR family protein [Populus trichocarpa] | 153 aa | XP_002324658.1 | GI: 224142631 |
| SAUR family protein [Populus trichocarpa] | 144 aa | XP_002323936.1 | GI: 224141147 |
| SAUR family protein [Populus trichocarpa] | 160 aa | XP_002323760.1 | GI: 224140787 |
| SAUR family protein [Populus trichocarpa] | 119 aa | XP_002323723.1 | GI: 224140713 |
| SAUR family protein [Populus trichocarpa] | 128 aa | XP_002323722.1 | GI: 224140711 |
| SAUR family protein [Populus trichocarpa] | 148 aa | XP_002323721.1 | GI: 224140709 |
| SAUR family protein [Populus trichocarpa] | 125 aa | XP_002323477.1 | GI: 224140209 |
| SAUR family protein [Populus trichocarpa] | 123 aa | XP_002323471.1 | GI: 224140197 |
| SAUR family protein [Populus trichocarpa] | 81 aa | XP_002327252.1 | GI: 224135575 |
| SAUR family protein [Populus trichocarpa] | 118 aa | XP_002321970.1 | GI: 224135047 |
| SAUR family protein [Populus trichocarpa] | 110 aa | XP_002320219.1 | GI: 224128011 |
| SAUR family protein [Populus trichocarpa] | 170 aa | XP_002320048.1 | GI: 224127334 |
| SAUR family protein [Populus trichocarpa] | 105 aa | XP_002329465.1 | GI: 224126755 |
| SAUR family protein [Populus trichocarpa] | 97 aa | XP_002319680.1 | GI: 224125808 |
| SAUR family protein [Populus trichocarpa] | 166 aa | XP_002329903.1 | GI: 224125140 |
| SAUR family protein [Populus trichocarpa] | 181 aa | XP_002318753.1 | GI: 224122102 |
| SAUR family protein [Populus trichocarpa] | 169 aa | XP_002330775.1 | GI: 224121228 |
| SAUR family protein [Populus trichocarpa] | 135 aa | XP_002318465.1 | GI: 224120972 |
| SAUR family protein [Populus trichocarpa] | 135 aa | XP_002331213.1 | GI: 224119654 |
| SAUR family protein [Populus trichocarpa] | 125 aa | XP_002317621.1 | GI: 224117608 |
| SAUR family protein [Populus trichocarpa] | 185 aa | XP_002316430.1 | GI: 224113231 |
| SAUR family protein [Populus trichocarpa] | 131 aa | XP_002316379.1 | GI: 224113065 |
| SAUR family protein [Populus trichocarpa] | 156 aa | XP_002315370.1 | GI: 224109962 |
| SAUR family protein [Populus trichocarpa] | 121 aa | XP_002315319.1 | GI: 224109812 |
| SAUR family protein [Populus trichocarpa] | 146 aa | XP_002313749.1 | GI: 224105269 |
| SAUR family protein [Populus trichocarpa] | 98 aa | XP_002313747.1 | GI: 224105265 |
| SAUR family protein [Populus trichocarpa] | 68 aa | XP_002313746.1 | GI: 224105263 |
| SAUR family protein [Populus trichocarpa] | 141 aa | XP_002313745.1 | GI: 224105261 |
| SAUR family protein [Populus trichocarpa] | 104 | XP_002313002.1 | GI: 224103303 |
| SAUR family protein [Populus trichocarpa] | 91 aa | XP_002313000.1 | GI: 224103297 |
| SAUR family protein [Populus trichocarpa] | 92 aa | XP_002312999.1 | GI: 224103295 |
| SAUR family protein [Populus trichocarpa] | 99 aa | XP_002312998.1 | GI: 224103293 |
| SAUR family protein [Populus trichocarpa] | 98 aa | XP_002312996.1 | GI: 224103287 |
| SAUR family protein [Populus trichocarpa] | 145 aa | XP_002312995.1 | GI: 224103283 |
| SAUR family protein [Populus trichocarpa] | 93 aa | XP_002312994.1 | GI: 224103279 |
| SAUR family protein [Populus trichocarpa] | 95 aa | XP_002312993.1 | GI: 224103277 |
| SAUR family protein [Populus trichocarpa] | 97 aa | XP_002312992.1 | GI: 224103275 |
| SAUR family protein [Populus trichocarpa] | 65 aa | XP_002312991.1 | GI: 224103273 |
| SAUR family protein [Populus trichocarpa] | 90 aa | XP_002312990.1 | GI: 224103269 |
| SAUR family protein [Populus trichocarpa] | 107 aa | XP_002312989.1 | GI: 224103265 |
| SAUR family protein [Populus trichocarpa] | 149 aa | XP_002312928.1 | GI: 224103107 |
| SAUR family protein [Populus trichocarpa] | 148 aa | XP_002312927.1 | GI: 224103103 |
| SAUR family protein [Populus trichocarpa] | 148 aa | XP_002312926.1 | GI: 224103101 |
| SAUR family protein [Populus trichocarpa] | 148 aa | XP_002312925.1 | GI: 224103099 |
| SAUR family protein [Populus trichocarpa] | 146 aa | XP_002312924.1 | GI: 224103097 |
| SAUR family protein [Populus trichocarpa] | 136 aa | XP_002311892.1 | GI: 224100477 |
| SAUR family protein [Populus trichocarpa] | 132 aa | XP_002311083.1 | GI: 224097848 |
| SAUR family protein [Populus trichocarpa] | 89 aa | XP_002334603.1 | GI: 224097550 |
| SAUR family protein [Populus trichocarpa] | 104 aa | XP_002310856.1 | GI: 224097158 |
| SAUR family protein [Populus trichocarpa] | 114 aa | XP_002310499.1 | GI: 224095881 |
| SAUR family protein [Populus trichocarpa] | 138 aa | XP_002310094.1 | GI: 224094216 |
| SAUR family protein [Populus trichocarpa] | 124 aa | XP_002309421.1 | GI: 224091969 |
| SAUR family protein [Populus trichocarpa] | 149 aa | XP_002308089.1 | GI: 224087160 |
| SAUR family protein [Populus trichocarpa] | 104 aa | XP_002307708.1 | GI: 224085816 |
| SAUR family protein [Populus trichocarpa] | 149 aa | XP_002306917.1 | GI: 224082982 |
| SAUR family protein [Populus trichocarpa] | 123 aa | XP_002306719.1 | GI: 224082502 |
| SAUR family protein [Populus trichocarpa] | 136 aa | XP_002306405.1 | GI: 224081427 |
| SAUR family protein [Populus trichocarpa] | 148 aa | XP_002306218.1 | GI: 224080736 |
| SAUR family protein [Populus trichocarpa] | 74 aa | XP_002306160.1 | GI: 224080554 |
| SAUR family protein [Populus trichocarpa] | 89 aa | XP_002306159.1 | GI: 224080550 |
| SAUR family protein [Populus trichocarpa] | 72 aa | XP_002306158.1 | GI: 224080548 |
| SAUR family protein [Populus trichocarpa] | 92 aa | XP_002306157.1 | GI: 224080546 |
| SAUR family protein [Populus trichocarpa] | 97 aa | XP_002306156.1 | GI: 224080544 |
| SAUR family protein [Populus trichocarpa] | 67 aa | XP_002306155.1 | GI: 224080540 |
| SAUR family protein [Populus trichocarpa] | 90 aa | XP_002306154.1 | GI: 224080536 |
| SAUR family protein [Populus trichocarpa] | 94 aa | XP_002306153.1 | GI: 224080532 |
| SAUR family protein [Populus trichocarpa] | 99 aa | XP_002306152.1 | GI: 224080530 |
| SAUR family protein [Populus trichocarpa] | 99 aa | XP_002306151.1 | GI: 224080528 |
| SAUR family protein [Populus trichocarpa] | 96 aa | XP_002306150.1 | GI: 224080524 |
| SAUR family protein [Populus trichocarpa] | 98 aa | XP_002306149.1 | GI: 224080522 |
| SAUR family protein [Populus trichocarpa] | 104 aa | XP_002306147.1 | GI: 224080516 |
| SAUR family protein [Populus trichocarpa] | 91 aa | XP_002305476.1 | GI: 224078022 |
| SAUR family protein [Populus trichocarpa] | 98 aa | XP_002305475.1 | GI: 224078016 |
| SAUR family protein [Populus trichocarpa] | 101 aa | XP_002305474.1 | GI: 224078010 |
| SAUR family protein [Populus trichocarpa] | 145 aa | XP_002305471.1 | GI: 224077988 |
| SAUR family protein [Populus trichocarpa] | 126 aa | XP_002304244.1 | GI: 224074081 |
| SAUR family protein [Populus trichocarpa] | 167 aa | XP_002303770.1 | GI: 224072526 |
| SAUR family protein [Populus trichocarpa] | 169 aa | XP_002303522.1 | GI: 224071573 |
| SAUR family protein [Populus trichocarpa] | 106 aa | XP_002303325.1 | GI: 224071003 |
| SAUR family protein [Populus trichocarpa] | 128 aa | XP_002326368.1 | GI: 224069539 |
| SAUR family protein [Populus trichocarpa] | 122 aa | XP_002326359.1 | GI: 224069496 |
| SAUR family protein [Populus trichocarpa] | 126 aa | XP_002326308.1 | GI: 224069236 |
| SAUR family protein [Populus trichocarpa] | 160 aa | XP_002326143.1 | GI: 224068552 |
| SAUR family protein [Populus trichocarpa] | 150 aa | XP_002302160.1 | GI: 224066607 |
| SAUR family protein [Populus trichocarpa] | 149 aa | XP_002301987.1 | GI: 224065947 |
| SAUR family protein [Populus trichocarpa] | 100 aa | XP_002301881.1 | GI: 224065607 |
| SAUR family protein [Populus trichocarpa] | 111 aa | XP_002301429.1 | GI: 224064344 |
| SAUR family protein [Populus trichocarpa] | 171 aa | XP_002301272.1 | GI: 224063729 |
| SAUR family protein [Populus trichocarpa] | 94 aa | XP_002300868.1 | GI: 224062657 |
| SAUR family protein [Populus trichocarpa] | 104 aa | XP_002300717.1 | GI: 224062025 |
| SAUR family protein [Populus trichocarpa] | 106 aa | XP_002300214.1 | GI: 224060467 |
| SAUR family protein [Populus trichocarpa] | 160 aa | XP_002299329.1 | GI: 224057798 |
| SAUR family protein [Populus trichocarpa] | 127 aa | XP_002299002.1 | GI: 224056745 |
| auxin-induced SAUR 1 [Antirrhinum majus] | 48 aa | AAL55415.1 | GI: 18071492 |
| SAUR family protein [Populus trichocarpa] | 89 aa | EEF10620.1 | GI: 222873489 |
| SAUR family protein [Populus trichocarpa] | 135 aa | EEF10465.1 | GI: 222873334 |
| SAUR family protein [Populus trichocarpa] | 169 aa | EEF09708.1 | GI: 222872577 |
| SAUR family protein [Populus trichocarpa] | 166 aa | EEF08271.1 | GI: 222871140 |
| SAUR family protein [Populus trichocarpa] | 105 aa | EEF07276.1 | GI: 222870145 |
| SAUR family protein [Populus trichocarpa] | 81 aa | EEE74057.1 | GI: 222835622 |
| SAUR family protein [Populus trichocarpa] | 128 aa | EEE72038.1 | GI: 222833561 |
| SAUR family protein [Populus trichocarpa] | 122 aa | EEE72029.1 | GI: 222833552 |
| SAUR family protein [Populus trichocarpa] | 126 aa | EEE71978.1 | GI: 222833501 |
| SAUR family protein [Populus trichocarpa] | 160 aa | EEE71813.1 | GI: 222833336 |
| SAUR family protein [Populus trichocarpa] | 109 aa | EEE77750.1 | GI: 222839413 |
| SAUR family protein [Populus trichocarpa] | 144 aa | EEE73392.1 | GI: 222834943 |
| SAUR12 [Zea mays] | 117 aa | ACG48743.1 | GI: 195658551 |
| SAUR33 [Zea mays] | 135 aa | ACG48640.1 | GI: 195658345 |
| SAUR40 [Zea mays] | 89 aa | ACG47423.1 | GI: 195655911 |
| SAUR1 [Zea mays] | 103 aa | ACG46362.1 | GI: 195653789 |
| SAUR52 [Zea mays] | 152 aa | ACG45294.1 | GI: 195651653 |
| SAUR36 [Zea mays] | 145 aa | ACG45154.1 | GI: 195651373 |
| SAUR37 [Zea mays] | 160 aa | ACG44891.1 | GI: 195650847 |
| SAUR11 [Zea mays] | 181 aa | ACG44091.1 | GI: 195649247 |
| SAUR23 [Zea mays] | 161 aa | ACG43656.1 | GI: 195648376 |
| SAUR30 [Zea mays] | 117 aa | ACG43481.1 | GI: 195648026 |
| SAUR55 [Zea mays] | 158 aa | ACG41822.1 | GI: 195644708 |
| SAUR20 [Zea mays] | 169 aa | ACG39599.1 | GI: 195640262 |
| SAUR44 [Zea mays] | 149 aa | ACG38928.1 | GI: 195638920 |
| SAUR56 [Zea mays] | 127 aa | ACG38150.1 | GI: 195637364 |
| SAUR55 [Zea mays] | 149 aa | ACG35907.1 | GI: 195628154 |
| SAUR31 [Zea mays] | 102 aa | ACG31378.1 | GI: 195619096 |
| SAUR25 [Zea mays] | 139 aa | ACG30255.1 | GI: 195616850 |
| SAUR14 [Zea mays] | 103 aa | ACG27349.1 | GI: 195611038 |
| SAUR16 [Zea mays] | 108 aa | ACG27315.1 | GI: 195610970 |
| SAUR25 [Zea mays] | 138 aa | ACG26269.1 | GI: 195608878 |
| SAUR9 [Zea mays] | 107 aa | ACG25834.1 | GI: 195608008 |
| SAUR_C [Arabidopsis thaliana] | 189 aa | NP_194860.1 | GI: 15235917 |
| SAUR_D [Arabidopsis thaliana] | 178 aa | NP_180016.1 | GI: 15224133 |
| SAUR_B [Arabidopsis thaliana] | 190 aa | NP_001031914.1 | GI: 79328260 |
| SAUR_E [Arabidopsis thaliana] | 150 aa | NP_195201.1 | GI: 15236186 |
| [Arabidopsis thaliana] | 160 aa | NP_189898.1 | GI: 15229222 |
| [Oryza sativa JaponicaC | 166 aa | BAG98524.1 | GI: 215766296 |
| [Oryza sativa Japonica Group] | 153 aa | BAG98478.1 | GI: 215766250 |
| [Oryza sativa Japonica Group] | 128 aa | BAG98236.1 | GI: 215766008 |
| [Oryza sativa Japonica Group] | 143 aa | BAG98212.1 | GI: 215765984 |
| [Oryza sativa Japonica Group] | 120 aa | BAG87305.1 | GI: 215765608 |
| [Oryza sativa Japonica Group] | 141 aa | BAG97924.1 | GI: 215741429 |
| [Oryza sativa Japonica Group] | 133 aa | BAG97805.1 | GI: 215741310 |
| [Oryza sativa Japonica Group] | 96 aa | BAG88609.1 | GI: 215693227 |
| [Arabidopsis thaliana] | 112 aa | NP_179392.1 | GI: 15227953 |
| [Arabidopsis thaliana] | 108 aa | NP_179248.1 | GI: 15227246 |
| [Arabidopsis thaliana] | 104 aa | NP_179718.1 | GI: 15226486 |
| [Arabidopsis thaliana] | 98 aa | NP_179717.1 | GI: 15226485 |
| [Arabidopsis thaliana] | 86 aa | NP_179716.1 | GI: 15226484 |
| [Arabidopsis thaliana] | 142 aa | NP_200171.2 | GI: 42568515 |
| [Arabidopsis thaliana] | 146 aa | NP_173471.2 | GI: 30686846 |
| [Arabidopsis thaliana] | 135 aa | NP_181163.2 | GI: 30686707 |
| [Arabidopsis thaliana] | 131 aa | NP_849679.1 | GI: 30685273 |
| [Arabidopsis thaliana] | 93 aa | NP_187035.2 | GI: 30678959 |
| [Arabidopsis thaliana] | 131 aa | NP_683527.1 | GI: 22330829 |
| [Arabidopsis thaliana] | 122 aa | NP_567196.1 | GI: 18411465 |
| [Arabidopsis thaliana] | 154 aa | NP_565113.1 | GI: 18410889 |
| [Arabidopsis thaliana] | 119 aa | NP_565042.1 | GI: 18410081 |
| [Arabidopsis thaliana] | 124 aa | NP_565665.1 | GI: 18401625 |
| [Arabidopsis thaliana] | 139 aa | NP_566440.1 | GI: 18399805 |
| SAUR68 [Arabidopsis thaliana] | 143 aa | NP_564332.1 | GI: 18397123 |
| [Arabidopsis thaliana] | 102 aa | NP_564331.1 | GI: 18397116 |
| [Arabidopsis thaliana] | 148 aa | NP_564330.1 | GI: 18397103 |
| [Arabidopsis thaliana] | 141 aa | NP_564329.1 | GI: 18397101 |
| [Arabidopsis thaliana] | 114 aa | NP_195951.1 | GI: 15242718 |
| [Arabidopsis thaliana] | 127 aa | NP_197582.1 | GI: 15242071 |
| [Arabidopsis thaliana] | 183 aa | NP_199889.1 | GI: 15241259 |
| [Arabidopsis thaliana] | 142 aa | NP_198130.1 | GI: 15241052 |
| [Arabidopsis thaliana] | 99 aa | NP_201427.1 | GI: 15239314 |
| [Arabidopsis thaliana] | 111 aa | NP_199056.1 | GI: 15238955 |
| [Arabidopsis thaliana] | 148 aa | NP_196660.1 | GI: 15238919 |
| [Arabidopsis thaliana] | 90 aa | NP_197309.1 | GI: 15238736 |
| [Arabidopsis thaliana] | 90 aa | NP_197307.1 | GI: 15238721 |
| [Arabidopsis thaliana] | 90 aa | NP_197306.1 | GI: 15238719 |
| [Arabidopsis thaliana] | 88 aa | NP_197304.1 | GI: 15238716 |
| [Arabidopsis thaliana] | 91 aa | NP_197303.1 | GI: 15238715 |
| [Arabidopsis thaliana] | 90 aa | NP_197302.1 | GI: 15238714 |
| [Arabidopsis thaliana] | 92 aa | NP_193115.1 | GI: 15236351 |
| [Arabidopsis thaliana] | 105 aa | NP_195207.1 | GI: 15236200 |
| [Arabidopsis thaliana] | 94 aa | NP_195206.1 | GI: 15236199 |
| [Arabidopsis thaliana] | 108 aa | NP_195205.1 | GI: 15236198 |
| [Arabidopsis thaliana] | 106 aa | NP_195204.1 | GI: 15236189 |
| [Arabidopsis thaliana] | 104 aa | NP_195203.1 | GI: 15236188 |
| [Arabidopsis thaliana] | 107 aa | NP_195202.1 | GI: 15236187 |
| [Arabidopsis thaliana] | 160 aa | NP_193993.1 | GI: 15235723 |
| [Arabidopsis thaliana] | 105 aa | NP_195597.1 | GI: 15234829 |
| SAUR15 [Arabidopsis thaliana] | 89 aa | NP_195596.1 | GI: 15234827 |
| [Arabidopsis thaliana] | 99 aa | NP_195595.1 | GI: 15234825 |
| [Arabidopsis thaliana] | 157 aa | NP_192978.1 | GI: 15234550 |
| [Arabidopsis thaliana] | 104 aa | NP_195334.1 | GI: 15234294 |
| [Arabidopsis thaliana] | 103 aa | NP_192691.1 | GI: 15233907 |
| [Arabidopsis thaliana] | 113 aa | NP_187598.1 | GI: 15232781 |
| [Arabidopsis thaliana] | 170 aa | NP_191628.1 | GI: 15232400 |
| [Arabidopsis thaliana] | 109 aa | NP_190893.1 | GI: 15231777 |
| [Arabidopsis thaliana] | 118 aa | NP_188657.1 | GI: 15231081 |
| [Arabidopsis thaliana] | 132 aa | NP_187889.1 | GI: 15230601 |
| [Arabidopsis thaliana] | 106 aa | NP_190688.1 | GI: 15230423 |
| [Arabidopsis thaliana] | 95 aa | NP_187034.1 | GI: 15228641 |
| [Arabidopsis thaliana] | 92 aa | NP_187033.1 | GI: 15228640 |
| [Arabidopsis thaliana] | 96 aa | NP_187032.1 | GI: 15228639 |
| [Arabidopsis thaliana] | 136 aa | NP_191749.1 | GI: 15228626 |
| [Arabidopsis thaliana] | 124 aa | NP_181240.1 | GI: 15228072 |
| [Arabidopsis thaliana] | 121 aa | NP_182192.1 | GI: 15226425 |
| [Arabidopsis thaliana] | 162 aa | NP_182046.1 | GI: 15225432 |
| [Arabidopsis thaliana] | 153 aa | NP_173413.1 | GI: 15223691 |
| [Arabidopsis thaliana] | 117 aa | NP_173411.1 | GI: 15223685 |
| [Arabidopsis thaliana] | 110 aa | NP_176011.1 | GI: 15223462 |
| [Arabidopsis thaliana] | 123 aa | NP_177746.1 | GI: 15222984 |
| [Arabidopsis thaliana] | 108 aa | NP_177688.1 | GI: 15222294 |
| [Arabidopsis thaliana] | 134 aa | NP_178034.1 | GI: 15219296 |
| [Arabidopsis thaliana] | 147 aa | NP_173100.1 | GI: 15219275 |
| [Arabidopsis thaliana] | 135 aa | NP_174243.1 | GI: 15218951 |
| [Arabidopsis thaliana] | 141 aa | NP_174236.1 | GI: 15218924 |
| [Arabidopsis thaliana] | 104 aa | NP_175002.1 | GI: 15218238 |
| Os09g0437100 [Oryza sativa Japonica] | 165 aa | BAH94585.1 | GI: 255678926 |
| Os06g0702000 [Oryza sativa Japonica] | 61 aa | BAH93710.1 | GI: 255677370 |
| Os06g0701900 [Oryza sativa Japonica] | 134 aa | BAF20401.2 | GI: 255677369 |
| Os06g0671150 [Oryza sativa Japonica] | 140 aa | BAH93678.1 | GI: 255677311 |
| Os04g0617050 [Oryza sativa Japonica] | 120 aa | BAH92818.1 | GI: 255675777 |
| Os04g0517900 [Oryza sativa Japonica] | 129 aa | BAH92744.1 | GI: 255675624 |
| Os01g0924966 [Oryza sativa Japonica] | 173 aa | BAH91446.1 | GI: 255674015 |
| Os01g0768333 [Oryza sativa Japonica] | 122 aa | BAH91311.1 | GI: 255673718 |
| Os02g0306200 [Oryza sativa Japonica] | 143 aa | BAH91643.1 | GI: 255670825 |
| Os02g0143400 [Oryza sativa Japonica] | 130 aa | BAF07783.2 | GI: 255670598 |
| Os02g0143300 [Oryza sativa Japonica] | 120 aa | BAF07782.2 | GI: 255670597 |
| Os12g0626200 [Oryza sativa Japonica] | 130 aa | BAF30342.1 | GI: 113649830 |
| Os10g0510500 [Oryza sativa Japonica] | 125 aa | BAF26951.1 | GI: 113639646 |
| Os09g0546100 [Oryza sativa Japonica] | 141 aa | BAF25753.1 | GI: 113632072 |
| Os09g0437400 [Oryza sativa Japonica] | 190 aa | BAF25178.1 | GI: 113631497 |
| Os08g0452500 [Oryza sativa Japonica] | 133 aa | BAF23868.1 | GI: 113623923 |
| Os08g0118500 [Oryza sativa Japonica] | 109 aa | BAF22782.1 | GI: 113622837 |
| Os07g0475700 [Oryza sativa Japonica] | 120 aa | BAF21536.1 | GI: 113611158 |
| Os06g0714300 [Oryza sativa Japonica] | 141 aa | BAF20487.1 | GI: 113596613 |
| Os04g0662400 [Oryza sativa Japonica] | 153 aa | BAF16070.1 | GI: 113565727 |
| Os04g0662200 [Oryza sativa Japonica] | 143 aa | BAF16069.1 | GI: 113565726 |
| Os02g0769100 [Oryza sativa Japonica] | 128 aa | BAF10155.1 | GI: 113537772 |
| Os02g0643800 [Oryza sativa Japonica] | 190 aa | BAF09475.1 | GI: 113537092 |
| Os02g0512000 [Oryza sativa Japonica] | 166 aa | BAF08854.1 | GI: 113536471 |
| Os02g0445600 [Oryza sativa Japonica] | 94 aa | BAF08663.1 | GI: 113536280 |
| Os02g0445100 [Oryza sativa Japonica] | 96 aa | BAF08662.1 | GI: 113536279 |
| [Oryza sativa Japonica Group] | 90 aa | BAH01591.1 | GI: 215769362 |
| [Oryza sativa Japonica Group] | 129 aa | BAH01004.1 | GI: 215768775 |
| [Oryza sativa Japonica Group] | 171 aa | BAH00993.1 | GI: 215768764 |
| [Oryza sativa Japonica Group] | 141 aa | BAH00901.1 | GI: 215768672 |
| [Oryza sativa Japonica Group] | 176 aa | BAG99230.1 | GI: 215767002 |
| [Oryza sativa Japonica Group] | 173 aa | BAG99136.1 | GI: 215766908 |
| [Oryza sativa Japonica Group] | 150 aa | BAG86730.1 | GI: 215765033 |
| [Oryza sativa Japonica Group] | 133 aa | BAG95554.1 | GI: 215734832 |
| [Oryza sativa Japonica Group] | 141 aa | BAG88424.1 | GI: 215693004 |
| [Arabidopsis thaliana] | 99 aa | CAB80547.1 | GI: 7270867 |
| Os12g0626200 [Oryza sativa] | 130 aa | NP_001067323.1 | GI: 115489672 |
| Os10g0510500 [Oryza sativa] | 125 aa | NP_001065037.1 | GI: 115482888 |
| Os09g0547000 [Oryza sativa] | 141 aa | NP_001063846.1 | GI: 115480505 |
| Os09g0546300 [Oryza sativa] | 157 aa | NP_001063841.1 | GI: 115480495 |
| Os09g0546000 [Oryza sativa] | 144 aa | NP_001063838.1 | GI: 115480489 |
| Os09g0508100 [Oryza sativa] | 138 aa | NP_001063619.1 | GI: 115480051 |
| Os09g0437400 [Oryza sativa] | 190 aa | NP_001063264.1 | GI: 115479341 |
| Os08g0452500 [Oryza sativa] | 133 aa | NP_001061954.1 | GI: 115476716 |
| Os08g0118500 [Oryza sativa] | 109 aa | NP_001060868.1 | GI: 115474543 |
| Os07g0475700 [Oryza sativa] | 120 aa | NP_001059622.1 | GI: 115472047 |
| Os06g0714300 [Oryza sativa] | 141 aa | NP_001058573.1 | GI: 115469948 |
| Os06g0701900 [Oryza sativa] | 312 aa | NP_001058487.1 | GI: 115469776 |
| Os04g0662400 [Oryza sativa] | 153 aa | NP_001054156.1 | GI: 115461112 |
| Os04g0662200 [Oryza sativa] | 143 aa | NP_001054155.1 | GI: 115461110 |
| Os04g0608300 [Oryza sativa] | 176 aa | NP_001053812.1 | GI: 115460424 |
| Os02g0769100 [Oryza sativa] | 128 aa | NP_001048241.1 | GI: 115448923 |
| Os02g0643800 [Oryza sativa] | 190 aa | NP_001047561.1 | GI: 115447563 |
| Os02g0512000 [Oryza sativa] | 166 aa | NP_001046940.1 | GI: 115446321 |
| Os02g0445600 [Oryza sativa] | 94 aa | NP_001046749.1 | GI: 115445939 |
| Os02g0445100 [Oryza sativa] | 96 aa | NP_001046748.1 | GI: 115445937 |
| Os02g0143400 [Oryza sativa] | 124 aa | NP_001045869.1 | GI: 115444179 |
| Os02g0143300 [Oryza sativa] | 121 aa | NP_001045868.1 | GI: 115444177 |
| [Vigna radiata var. radiata] | 92 aa | BAA03310.1 | GI: 287570 |
| [Vigna radiata var. radiata] | 196 aa | BAA03309.1 | GI: 287568 |
| [Vigna radiata var. radiata] | 194 aa | BAA03308.1 | GI: 287566 |
| SAUR-AC-like protein [Arabidopsis thaliana] | 92 aa | CAB78421.1 | GI: 7268083 |

### BRIEF DESCRIPTION OF THE FIGURES

FIG. 1: Hypocotyl length of 10 days old (do) light grown seedlings. N-terminal fusions of GFP or StrepII with SAUR19 and SAUR24 confer increased hypocotyl length (1) Col control; (2) N-terminal GFP-SAUR 19 fusion; (3) N-terminal StrepII-SAUR19 fusion; (4) N-terminal StrepII-SAUR24 fusion.

FIG. 2: Hypocotyl length of 10 do light grown seedlings. N-terminal GFP fusions of SAUR19, 21, 23, or 24 confer increased hypocotyl length. Untagged or C-terminally tagged SAUR19 constructs resemble Col controls. (1) Col control; (2) N-terminal GFP-SAUR19 fusion; (3) N-terminal GFP-SAUR21 fusion; (4) N-terminal GFP-SAUR23 fusion; (5) N-terminal GFP-SAUR24 fusion; (6) overexpression of non-fused SAUR19; (7) C-terminal SAUR19-GFP fusion

FIG. 3: Biomass measurements. The vegetative part of plants (Col Controls and GFP-SAUR19 plants) grown in vitro was harvested 20 days after stratification and fresh weigth was measured (n=12). Two independent lines are analyzed (SAUR19-1 and SAUR19-2)

FIG. 4: Leaf area measurements. Plants were grown in vitro for 20 days and leaf series were done to measure individual leaf area. Green: GFP-SAUR19 OE lines (two independent lines: SAUR19-1 and SAUR19-2), orange: wild type plants.

FIG. 5: Combination of GFP-SAUR19 overexpression with other IYG lines. N example of a combination leading to an additive effect on leaf area. B/example of a combination leading to a positive effect (more than expected) on growth. For a given cross, the leaf area expected in case of an additive effect (*) was calculated by adding the areas of the two heterozygous parents and subtracting the area from the control Col-0.

### EXAMPLES

### Materials and Methods to the Examples

### SAUR DNA Constructs

SAUR coding sequences were PCR amplified from Arabidopsis Col-0 genomic DNA and Gateway cloned into pDONR207. The resulting donor clones were sequenced and then used to transfer the SAUR coding regions into destination vectors pMDC32 (Curtiss and Grossniklaus, 2003) (35S::SAUR), pMDC43(Curtiss and Grossniklaus, 2003) (35S::GFP-SAUR), pMDC84(Curtiss and Grossniklaus, 2003) (35S::SAUR-GFP), or pJ2B-StepII-GW (35S::StrepII-SAUR) as per the Gateway LR Clonase protocol (Invitrogen). The resulting binary vectors were introduced into Agrobacterium strain GV3101, which was used to transform Arabidopsis Col-0 plants using the floral dip method.

### Plant Material and Growth Condition

Two independent homozygous lines (SAUR19-1 and SAUR19-2) overexpressing the SAUR19 (At5g18010) gene fused to the GFP at its N-terminal end were selected after transformation of agrobacterium with the construct containing the GFP-SAUR19 fusion under the control of the CaMV35S promoter. These plants were grown for phenotypic analysis in vitro in half-strength Murashige and Skoog medium (Murashige and Skoog, 1962), supplemented with 1% sucrose at 21° C. under a 16-h day/8-h night regime.

### Growth Analysis

For the biomass measurement, the vegetative part of a 20 days old plant is harvested and fresh weight is measured.

For the rosette leaf area measurements, 8-12 seedlings were grown under in vitro conditions for 20 days. Individual leaves (cotyledons and rosette leaves) were dissected and their area was measured with the ImageJ software (http://rsb.info.nih.gov/ij/).

### Hypocotyl Assays

Homozygous transgenic lines were identified and transgene expression was confirmed by Northern hybridization. Seeds from homozygous transgenic lines were sterilized with 30% bleach, stratified at 4° C. for four days, and then transferred to ATS (Lincoln et al., 1990) agar plates. Seedlings were grown for 10 days in a plant growth chamber at 20° C. under long day (16:8) light conditions. On day 10, seedlings were photographed and hypocotyl lengths measured on a computer using imaging software.

### Combination of Growth Enhancing Lines

To obtain the double trangenic plants, homozygous lines expressing the respective growth enhancing genes were crossed with each other and the F1 progeny analyzed. As controls, crosses were made with a Col-0 wild-type. Leaf series were performed from plants grown under in vitro conditions for 20 days and leaf area was measured with the ImageJ software (http://rsb.info.nih.gov/ij/).

### Example 1

### Phenotypic Analysis of Overexpressors of SAUR Fusion Proteins

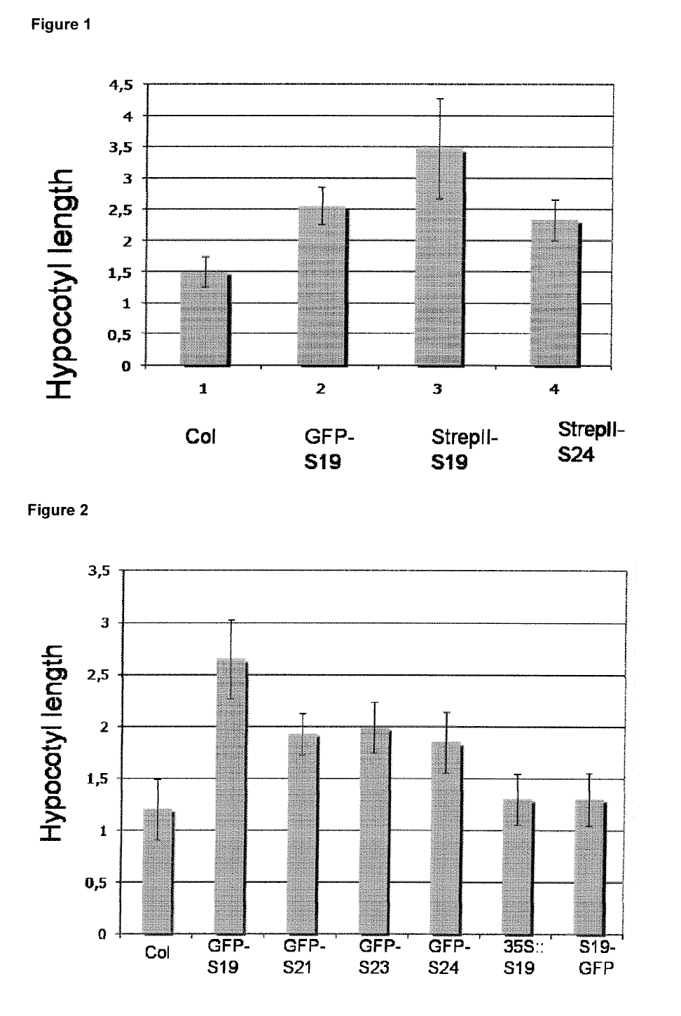

Plants expressing N-terminally tagged SAUR fusion proteins exhibited a 1.5-2× increase in hypocotyl length. This increase in organ size appears to be predominantly due to increased cell expansion, as measurements of cell length were highly correlated with hypocotyl length. In contrast, no effects on hypocotyl length were observed with plants overexpressing untagged or C-terminally-tagged SAUR proteins. A minimum of three independent transgenic lines were analyzed for each construct (FIGS. 1 and 2).

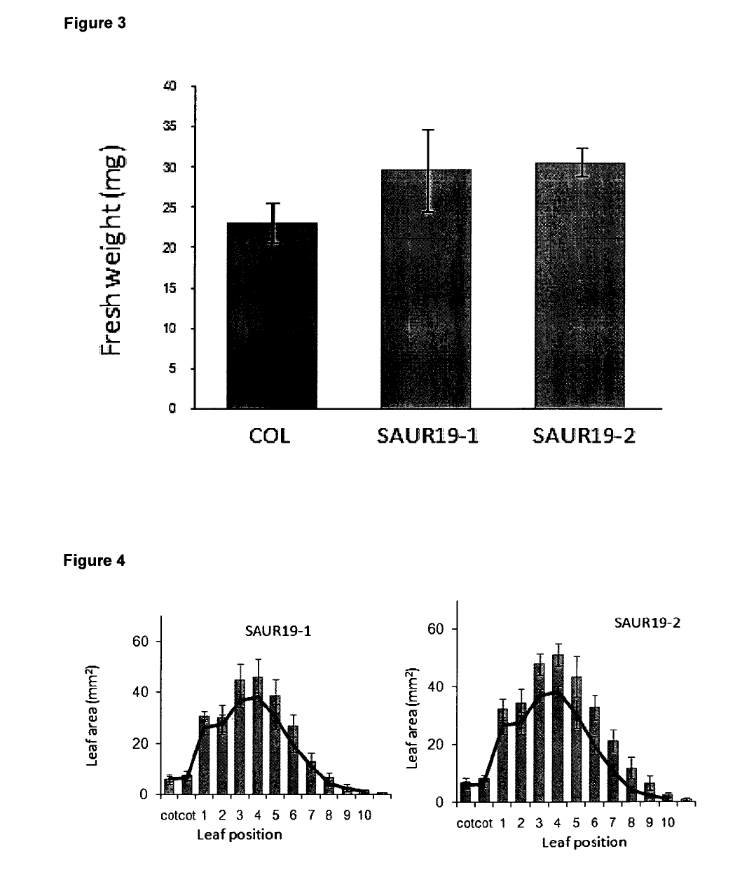

In order to evaluate the effect of the over-expression of SAUR19 on growth, measurements of biomass and leaf area were performed on the GFP-SAUR19 lines. The vegetative part of plants grown under in vitro conditions was harvested 20 days after stratification and fresh weight was measured. As shown in FIG. 3, the biomass of the GFP-SAUR19 is increased by 30% when compared to the wild type plants. Subsequently leaf series were made from plants harvested at the same stage in order to measure the area of each individual leaf from the rosette (FIG. 4). This analysis revealed that the two SAUR19-GFP lines lead to the production of larger leaves, however, the extent of the increased size differs between the two lines. The GFP-SAUR19 (line 1) leads to an increase of the area of leaves 4 to 6 while in GFP-SAUR19 (line 2), almost all the leaves are larger (2 to 9).

### Example 2

### Crosses with Other IYG Lines

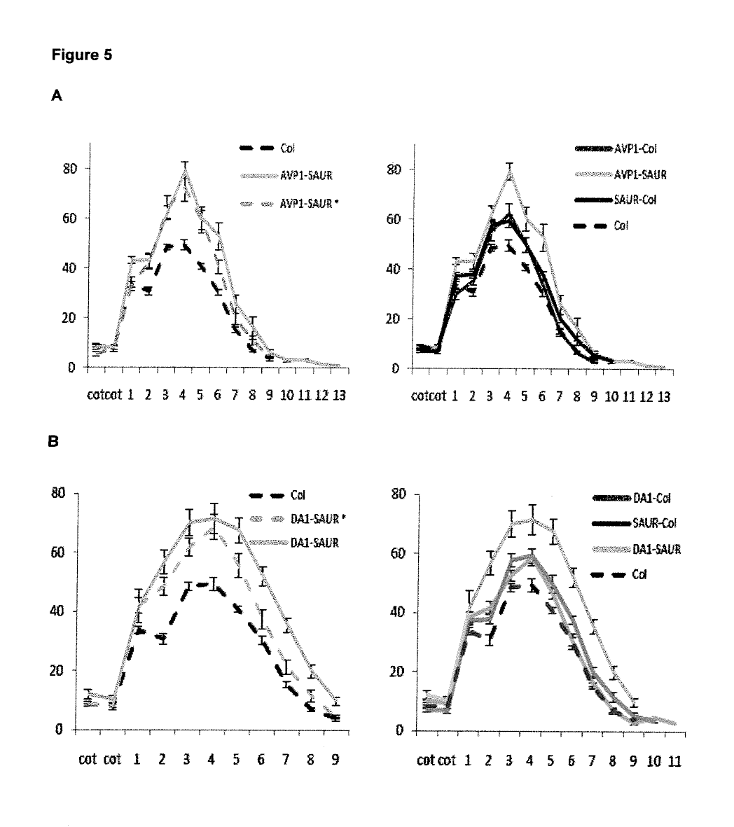

In order to investigate the potential genetic connections between SAUR19 and other genes leading to an increase in leaf area when over-expressed or down-regulated, crosses between GFP-SAUR19-2 and 10 Intrinsic Yield Genes (IYG) lines (AGF1 (WO02079403), ANT (Mizukami et al., 2000), APC10, ARL (Hu Y, et al. 2006), AVP1 (Li J, et al. 2005), BRI1 (Wang Z-Y, et al. 2001), DA1(Li Y, et al. 2008) EXP10 (Cho H-T, Cosgrove D J. 2000), GA20OX (Coles JP, et al. 1999), GRF5 (Horiguchi, et al. 2005) and JAW (Schommer C, et al. 2008)) were performed. IYG are genes that when overexpressed or mutated enhance plant growth. Growth behaviour of the heterozygous progeny was analyzed under standard conditions by measuring leaf area. This analysis allowed identifying additive/synergistic/antagonistic effects of gene combinations on growth. For example, when GFP-SAUR19 and AVP1 simultaneously are overexpressed in the same plant, the final leaf size corresponds to the sum of the effect of the heterozygous parents suggesting that these genes work independently to control organ size (FIG. 5A). Interestingly, the combination GFP-SAUR19 and DA1-1 leads to the production of leaves that are larger than what is expected for an additive effect, suggesting that these two genes work synergistically to increase leaf size (FIG. 5B). In most of the cases the combination leads to an additive effect (AVP1, APC10, AGF1, ANT, ARL, JAW, GRF5). In one case, GFP-SAUR19 combined with BRI, we observed a negative effect on leaf growth. For two cases, the combination of GFP-SAUR19 with EXP10 and GA20-ox, the effect on growth is unclear.

### REFERENCES

Anai, T., Kono, N., Kosemura, S., Yamamura, S. and Hasegawa, K. (1998). Isolation and characterization of an auxsin-inducilbe SAUR gene from radish seedlings. DNA seq. 9, 329-333.
Cho H T, Cosgrove D J (2000) Altered expression of expansin modulates leaf growth and pedicel abscission in Arabidopsis thaliana. Proc Natl Acad Sci USA 97: 9783-9788.
Coles J P, Phillips A L, Croker S J, Garcia-Lepe R, Lewis M J, Hedden P (1999) Modification of gibberellin production and plant development in Arabidopsis by sense and antisense expression of gibberellin 20-oxidase genes. Plant J 17: 547-556.
Curtis, M. D. and Grossniklaus, U. (2003). A gateway cloning vector set for high-throughput functional analysis of genes in planta. Plant Physiol 133, 462-469.
Franco, A. R., Gee, M. A. and Guilfoyle, T. J. (1990). Induction and superinduction of auxin-responsive mRNAs with auxin and protein synthesis inhibitors. J. Biol. Chem. 265, 15845-15849.
Gonzalez, N. Gerrit T S Beemster, G. T. S. and Inzé D. (2009).David and Goliath: what can the tiny weed Arabidopsis teach us to improve biomass production in crops? Current Opinion in Plant Biology. 12: 157-164.
Horiguchi G, Kim G T, Tsukaya H (2005) The transcription factor AtGRF5 and the transcription coactivator AN3 regulate cell proliferation in leaf primordia of Arabidopsis thaliana. Plant J 43: 68-78.
Hu Y, Poh H M, Chua N H (2006) The Arabidopsis ARGOS-LIKE gene regulates cell expansion during organ growth. Plant J 47: 1-9.
Jain, M., Tyagi, A. K. and Khurana, J. P. (2006). Genome-wide analysis, evolutionary expansion, and expression of early auxin-responsive SAUR gene family in rice (Oryzae sativa). Genomics, 88, 360-371.
Kant, S., Bi, Y. M., Zhu, T and Rothstein, S. J. (2009). SAUR39, a small auxin-up RNA gene, acts as a negative regulator of auxin synthesis and transport in rice. Plant Physiol. 151, 691-701.
Li J, Yang H, Peer W A, Richter G, Blakeslee J, Bandyopadhyay A, Titapiwantakun B, Undurraga S, Khodakovskaya M, Richards E L, Krizek B, Murphy A S, Gilroy S, Gaxiola R (2005) Arabidopsis H+-PPase AVP1 regulates auxin-mediated organ development. Science 310: 121-125.
Li Y, Zheng L, Corke F, Smith C, Bevan M W (2008) Control of final seed and organ size by the DA1 gene family in Arabidopsis thaliana. Genes Dev 22: 1331-1336.
Knauss, S., Rohrmeier, T. and Lehle, L. (2003). The auxin-induced maize gene ZmSAUR2 encodes short-lived nuclear protein expressed in elongating tissues.
Lincoln, C., Britton, J. H. and Estelle, M. (1990). Growth and development of the axrl mutants of Arabidopsis. Plant Cell 2, 1071-1080.
Marchler-Bauer, A., Anderson, J. B., Chitsaz, F., Derbyshire, M. K., DeWeese-Scott, C., Fong, J. H., Geer, L. Y., Geer, R. C., Gonzales, N. R., Gwadz, M., He, S., Hurwitz, D. I., Jackson, J. D., Ke, Z., Lanczycki, C. J., Liebert, C. A., Liu, C., Lu, F., Lu, S., Marchler, G. H., Mullokandov, M., Song, J. S., Tasneem, A., Thanki, N., Yamashita, R. A., Zhang, D., Zhang, N. and Bryant, S. H. (2009). CDD: specific functional annotation with the Conserved Domain Database. Nucleic Acids Res. 37(Database issue):D205-10.
Mizukami Y, Fischer R L (2000) Plant organ size control: AINTEGUMENTA regulates growth and cell numbers during organogenesis. Proc Natl Acad Sci USA 97: 942-947.
Murashige T, Skoog F (1962) A revised medium for rapid growth and bio assays with tobacco tissue cultures. Physiol Plant 15: 473 497
Palatnik J F, Allen E, Wu X, Schommer C, Schwab R, Carrington J C, Weigel D (2003) Control of leaf morphogenesis by microRNAs. Nature 425: 257-263.
Van Camp, W. (2005). Yield enhancement genes: seeds for growth. Curr Opin Biotech 16: 147-153
Wang Z Y, Seto H, Fujioka S, Yoshida S, Chory J (2001) BRI1 is a critical component of a plasma-membrane receptor for plant steroids. Nature 410: 380-383.

1. A fusion protein comprising a SAUR polypeptide and a heterologous polypeptide.

2. The fusion peptide according to claim 1, wherein said heterologous polypeptide is fused to the N-terminal end of the SAUR polypeptide.

3. The fusion protein according to claim 1, wherein said SAUR polypeptide comprises
(SEQ ID [[N^{o}]] NO: 5)
(E/K/R) G (H/N/F/Y/S) (V/L/F) (P/A) V (Y/S/C) V
and/or
(SEQ ID [[N^{o}]] NO: 6)
L L (R/E/K/S) (R/K/E/D) (A/V) (A/E) (Q/E) E (Y/F)
G (Y/F).

4. The fusion protein according to claim 3, wherein said SAUR polypeptide is selected from the group consisting of SAUR19, SAUR 21, SAUR 23 and SAUR24.

5. The fusion protein of claim 1, wherein said fusion protein comprises a peptide selected from the group consisting of SEQ ID NO:1, SEQ ID NO:2, SEQ ID NO:3, and SEQ ID NO:4.

6. A method of increasing plant growth and/or plant yield in a plant, the method comprising:
utilizing the fusion protein of claim 1 to increase plant growth and/or plant yield in the plant.

7. The method according to claim 6, wherein said fusion protein is expressed in combination with a protein selected from the group consisting of ARL, ANT, AGF1, APC10, GRF5, AVP1, a homologue thereof, and an orthologue thereof.

8. The method according to claim 6, wherein said fusion protein is expressed in combination with a downregulated or inactivated DA1-1 gene, or a homologue or orthologue thereof.

9. The method according to claim 6, wherein said fusion protein is expressed in combination with the expression of a microRNA encoded by JAW, or a homologue or orthologue thereof.

10. A transgenic plant, comprising the fusion protein of claim 1.

11. The transgenic plant according to claim 10, further comprising a recombinant gene encoding an inactive DA1-1 and/or a protein selected from the group consisting of ARL, ANT, AGF1, APC10, GRF5, AVP1, a homologue thereof, and an orthologue thereof.

12. The transgenic plant according to claim 10, further comprising a recombinant JAW gene encoding a microRNA, a homologue thereof, or an orthologue thereof.

13. The transgenic plant of claim 10, wherein said plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis.

14. A method for obtaining a plant with increased growth characteristics over wild-type plant, the method comprising:
isolating a nucleic acid encoding a polypeptide of the SAUR family,
fusing said nucleic acid at the 5′ end to a nucleic acid encoding a heterologous peptide, wherein said fusion results in a N-terminal fusion to the SAUR polypeptide; and
transforming the fused nucleic acid into a plant,
so as to obtain a plant having increased growth characteristics.

15. The method according to claim 14, wherein said plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis.

16. The fusion protein of claim 2, wherein the SAUR polypeptide comprises:
(SEQ ID NO: 5)
(E/K/R) G (H/N/F/Y/S) (V/L/F) (P/A) V (Y/S/C) V
and/or
(SEQ ID NO: 6)
L L (R/E/K/S) (R/K/E/D) (A/V) (A/E) (Q/E) E (Y/F)
G (Y/F).

17. The fusion protein of claim 16, wherein the SAUR polypeptide is selected from the group consisting of SAUR 19, SAUR 21, SAUR 23, and SAUR24.

18. The fusion protein of claim 2 comprising a peptide selected from the group consisting of SEQ ID NO:1, SEQ ID NO:2, SEQ ID NO:3, and SEQ ID NO:4.

19. The fusion protein of claim 3 comprising a peptide selected from the group consisting of SEQ ID NO:1, SEQ ID NO:2, SEQ ID NO:3, and SEQ ID NO:4.

20. The fusion protein of claim 4 comprising a peptide selected from the group consisting of SEQ ID NO:1, SEQ ID NO:2, SEQ ID NO:3, and SEQ ID NO:4.


SEQUENCE LISTING (6 sequences)

SEQ ID NO: 1 - protein, 90 aa, Arabidopsis thaliana
MAFVRSLLGAKKILSRSTAAGSAAPKGFLAVYVGESQKKRYLVPLSYLSQPSFQALLSKSEEEFGFAHPMGGLTIPCPEDTFINVTSRLQ

SEQ ID NO: 2 - protein, 88 aa, Arabidopsis thaliana
MALVRSLLGAKKILSRSTASAAPKGFLAVYVGESQKKRYLVPLSYLSQPSFQALLSKSEEEFGFDHPMGGLTIPCPEDTFINVTSRLQ

SEQ ID NO: 3 - protein, 90 aa, Arabidopsis thaliana
MALVRSLLVAKKILSRSAAAVSAPPKGFLAVYVGESQKKRYLVPLSYLNQPSFQALLSKSEEEFGFDHPMGGLTIPCPEDTFINVTSRLH

SEQ ID NO: 4 - protein, 90 aa, Arabidopsis thaliana
MAFVRSLLGAKKILSRSTGAGSAAPKGFLAVYVGESQKKRYLVPVSYLNQPSFQALLSKSEEEFGFDHPMGGLTIPCPEDTFINVTSRLQ

SEQ ID NO: 5 - protein, 8 aa, Arabidopsis thaliana
  MISC_FEATURE (1)..(1): Xaa can be E, K or R
XGXXXVXV

SEQ ID NO: 6 - protein, 11 aa, Arabidopsis thaliana
  MISC_FEATURE (3)..(3): Xaa can be R, E, K or S
LLXXXXXEXGX